(19) United States
(12) Patent Application Publication
(10) Pub. No.: US 2022/0362501 A1
(43) Pub. Date: Nov. 17, 2022

(54) AIR FLOW SYSTEM CANNULA

(71) Applicant: Invent Medical Corporation, Carlsbad, CA (US)

(72) Inventors: Nhien Nguyen, Vista, CA (US); Steve Han, Fountain Valley, CA (US); Samuel Chang, Carlsbad, CA (US)

(21) Appl. No.: 17/741,641

(22) Filed: May 11, 2022

Related U.S. Application Data
(60) Provisional application No. 63/187,222, filed on May 11, 2021.
(60) Provisional application No. 63/187,238, filed on May 11, 2021.
(60) Provisional application No. 63/187,255, filed on May 11, 2021.
(60) Provisional application No. 63/187,265, filed on May 11, 2021.

(51) Int. Cl. A61M 16/06; A61M 16/08

(52) U.S. Cl. CPC A61M 16/0666; A61M 16/0605; A61M 16/0683; A61M 16/0875

(57) ABSTRACT

An air flow system cannula is disclosed. The air flow system cannula comprises: a body having a radius of curvature less than about 180 degrees, the body including two solid portions and an adjustable portion provided between the two solid portions for additional flex and angular contouring, two nasal posts, and two end portions. The adjustable portion can collapse to a plurality of positions. The air flow system cannula can also include an adjustable frame that provides additional structural integrity. The adjustable frame is inserted into the body, snap-fit to the body, pressed-fit to the body, or directly molded in the body.

### RELATED APPLICATIONS INFORMATION

The present application claims the benefit of priority under 35 U.S.C. 119(e) to Provisional Patent Application Ser. No. 63/187,222, entitled “AIR FLOW SYSTEM CANNULA WITH REDUCE RADIUS OF CURVATURE”, filed May 11, 2021, to Provisional Patent Application Ser. No. 63/187,238, entitled “AIR FLOW SYSTEM CANNULA WITH A COLLAPSIBLE PORTION”, filed May 11, 2021, Provisional Patent Application Ser. No. 63/187,255, entitled “AIR FLOW CANNULA WITH A BELLOWS PORTION”, filed May 11, 2021, and Provisional Patent Application Ser. No. 63/187,265, entitled “AIR FLOW SYSTEM CANNULA WITH A COLLAPSIBLE PORTION AND A ADJUSTABLE FRAME”, filed May 11, 2021, each of which is incorporated herein by reference as if set forth in full.

### TECHNICAL FIELD

The embodiments described herein are generally directed to air flow system cannulas, and, more particularly, to such cannulas that provide a more effective and consistent interface with the patient.

### BACKGROUND

Flow generation systems can be generally described as systems that generate a gaseous flow, for example airflow or a blend of ambient air and oxygen. A ventilator is one example of a flow generation system. A ventilator is a piece of medical equipment that delivers a flow of gas, such as a blend of oxygen and ambient air to the airway of a patient to assist in or substitute a patient's breathing. Most ventilators deliver a blend of oxygen and air so that the patient receives a target oxygen concentration greater than that of ambient air. Generally, ventilators utilize a combination of single-use or reusable disposable components for the patient interface (e.g. a mask or mouthpiece connected to flexible tubing) and non-disposable capital equipment (e.g. air pumps, sensors, controller modules, humidifiers, etc.) that is used over a period of time among different patients. The patient interface can be for example a mouthpiece, mask (full face, nasal, pillow, total mask, or combinations of these), endotracheal tube or tracheostomy tube.

Although there are a variety of ventilator designs currently used in the field, most conventional designs will fall into either the single-limb or double-limb category. Single-limb ventilators typically come in different configurations. In certain types of single limb configurations, there is no “active” exhalation valve. Instead, a hole (or multiple holes) at or near the patient connection serves as a “passive” exhalation valve. However, in this configuration, since the hole(s) is not big enough to handle the entire exhalation flow, some of the exhaled flow travels back to the device. In an acute care single-limb circuit, a single tube is also used for inhalation and exhalation. Typically, a section of the tube near the patient's mouthpiece is equipped with an exhalation valve, which is switched on and off according to a pressure and/or flow signal measured by the system. The pressure and/or flow signal can detect when air is flowing from the ventilator equipment to the patient, causing the exhalation valve to stay closed. The pressure and/or flow signal can also detect when air stops flowing, or when an upstream airflow is detected, causing the exhalation valve to open. Double limb circuits are similar, except that they have a second tube connecting back to the ventilator, where the exhalation valve is located in this case. The advantage of a single limb circuit is that it eliminates the issues of added bulk, weight and production costs that are present with double limb design. However, one of the shortcomings of single-limb ventilators is cross-contamination of the ventilator, since exhaled gas from the patient can return to the dedicated or non-disposable components of the ventilator system during exhalation.

U.S. Pat. No. 10,525,222, which is incorporated herein by reference in its entirety as if set forth in full, discloses novel systems and methods for preventing such cross-contamination. But another issue with conventional flow generation systems is inefficient design of the cannula, which interfaces the tubing, and therefore the air flow to the patients nose.

### SUMMARY

According to one aspect, an air flow cannula is disclosed. The air flow cannula comprises a body having a radius of curvature less than about 180 degrees, the body including two solid portions and an adjustable portion between the two solid portions, two nasal posts, and two end portions. The air flow cannula can include an adjustable frame attached to the body and configured to provide additional structural integrity to the air flow cannula.

According to another aspect, a tube system is disclosed. The tube system comprises tubing; a head strap; an air flow system cannula; a cannula plug; and a cannula tube fitting. The air flow system cannula comprises a body having a radius of curvature less than about 180 degrees, the body comprising two solid portions and an adjustable portion between the two solid portions, two nasal posts, each of the two nasal posts protruding from a respective one of the two solid portions of the body, and two end portions, each of the two end portions comprising a hole and a window. The cannula plug comprises a first cannula plug tab configured to engage the window in one of the two end portions, a second cannula plug tab configured to enable the cannula plug to be pushed into the window of the same end portion, and a coupling mechanism for coupling the cannula plug with the head strap. The cannula tube fitting comprises a first cannula tube fitting tab at a first end of the cannula tube fitting configured to engage the window in the other of the two end portions, a second cannula tube fitting tab configured to enable the cannula tube fitting to be pushed into the window of the same end portion, a coupling mechanism for coupling the cannula plug with the head strap, and a second end of the cannula tube fitting configured to couple with the tubing.

### BRIEF DESCRIPTION OF THE DRAWINGS

The details of embodiments of the present disclosure, both as to their structure and operation, may be gleaned in part by study of the accompanying drawings, in which like reference numerals refer to like parts, and in which:

FIGS. 1A and 1B illustrate an air flow system cannula, according to an example embodiment;

FIGS. 2A and 2B illustrate a tubing system that comprises the cannula of FIGS. 1A and 1B, according to an example embodiment;

FIGS. 3A and 3B illustrate an air flow system cannula, according to another example embodiment;

FIG. 3C illustrates an air flow system cannula, according to another example embodiment;

FIG. 3D illustrates an air flow system cannula, according to another example embodiment;

FIG. 4 illustrates a tubing system that comprises the cannula of FIGS. 3A and 3B, according to an example embodiment;

FIG. 5 illustrates an air flow system cannula, according to another example embodiment;

FIG. 6 illustrates a tubing system that comprises the cannula of FIG. 5, according to an example embodiment;

FIGS. 7A and 7B illustrate an air flow system cannula, according to an example embodiment;

FIG. 8 illustrates an air flow system cannula, according to another example embodiment; and

FIG. 9 illustrates an air flow system cannula, according to another example embodiment.

### DETAILED DESCRIPTION

The detailed description set forth below, in connection with the accompanying drawings, is intended as a description of various embodiments, and is not intended to represent the only embodiments in which the disclosure may be practiced. The detailed description includes specific details for the purpose of providing a thorough understanding of the embodiments. However, it will be apparent to those skilled in the art that embodiments of the invention can be practiced without these specific details. In some instances, well-known structures and components are shown in simplified form for brevity of description.

For clarity and ease of explanation, some surfaces and details may be omitted in the present description and figures. In addition, references herein to “upstream” and “downstream” or “forward” and “aft” are relative to the flow direction of the primary gas (e.g., air) used in the combustion process, unless specified otherwise. It should be understood that “upstream,” “forward,” and “leading” refer to a position that is closer to the source of the primary gas or a direction towards the source of the primary gas, and “downstream,” “aft,” and “trailing” refer to a position that is farther from the source of the primary gas or a direction that is away from the source of the primary gas. Thus, a trailing edge or end of a component (e.g., a turbine blade) is downstream from a leading edge or end of the same component. Also, it should be understood that, as used herein, the terms “side,” “top,” “bottom,” “front,” “rear,” “above,” “below,” and the like are used for convenience of understanding to convey the relative positions of various components with respect to each other, and do not imply any specific orientation of those components in absolute terms (e.g., with respect to the external environment or the ground).

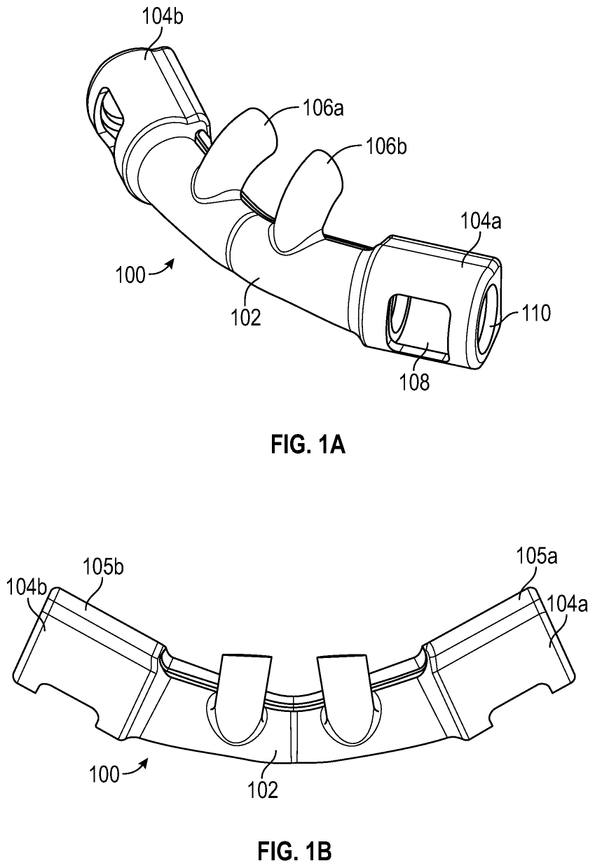

FIG. 1A illustrates an example cannula 100 in accordance with one example embodiment. As can be seen, cannula 100 comprises a body 102 and end portions 104a and 104b and prongs 106a and 106b, which interface the cannula with the patient's nose. Prongs 106a and 106b, should fill less than 50% of the patient's nostrils. It will be understood that the prongs need to be effectively interfaced with the patients nose in order to ensure a more optimum air flow to the patient. Conventional designs are not as effective as they could be at providing the effective and consistent interface. One reason for this is that the radius of curvature of the body of conventional cannulas is too large, i.e., the cannulas are too flat. As such when the cannula is attached to a head strap and interfaced with the patient's head, the cannula may not provide sufficient coupling, because the prongs are not sufficiently couple with het patient's nose.

In the example of FIG. 1A, the radius of curvature is smaller so that it more effectively couples with patients with smaller heads or less distance between their nostrils. At the same time, the cannula can be made of flexible material such that is can stretch and flatten out for patients with larger heads or a greater distance between their nostrils.

In certain embodiment, the radius of curvature for the cannula 100 is less than 180, and in particular less than 175.

Another problem with conventional cannulas is that they provide poor coupling with the tubing. Cannula 100 comprises improved end portions 104a and 104b that allow for improved connection or coupling with the tubing, which is described in more detail below. As can be seen, each end portion 104a and 104b has a hole 110 for receiving a cannula tube fitting as described below. Each end portion 104a and 104b also comprises a window 108 that can accommodate a tab on the cannula tube fitting for effective coupling of the fitting with the end portion 104a and 104b.

Because the cannula 100 comprises matching end portions 104a and 104b, the tubing can be interfaced with either end of the cannula. In various exemplary embodiments, the cannula 100, where it is configured to contact skin, includes one or more skin interface portions 105a/105b, for example, one or more of: a cooling pad, gel, and breathable foam pad. In one embodiment, the pad or gel is a single continuous skin interface portion. In another embodiment, the cannula 100 includes two or more skin interface portions (for example, a first skin interface portion 105b at the first end portion 104a and a second skin interface portion 105b at the second end portion 104b).

FIG. 1B is a top view of the cannula 100.

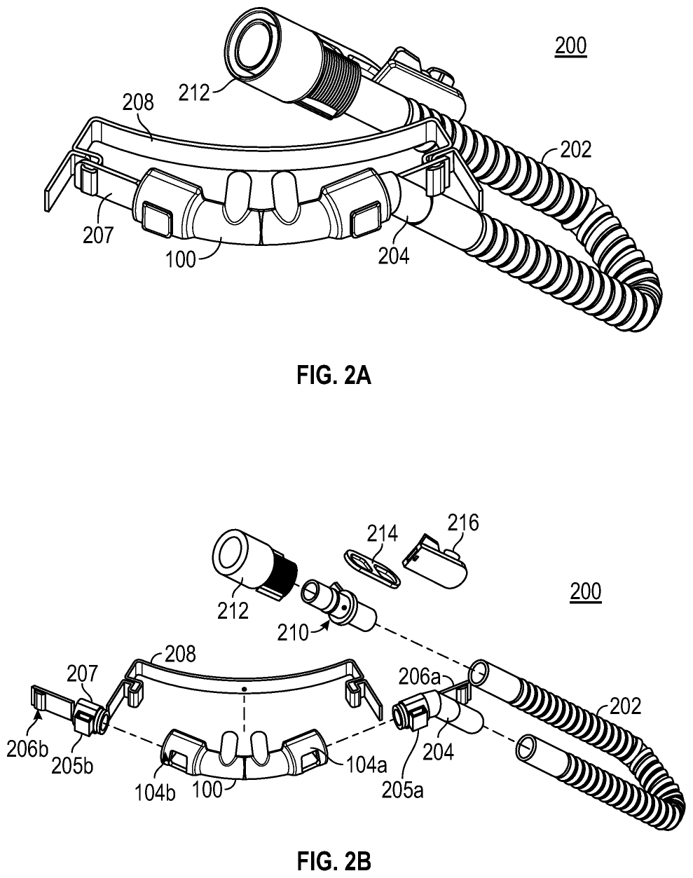

FIGS. 2A and 2B illustrate a tubing assembly 200 that includes the cannula 100. As can be seen better in the exploded view of FIG. 2A, assembly 200 comprises, in addition to cannula 100, tubing 202, which can be couple with cannula 100 via rigid cannula tube fitting 204. Fitting 204 can comprise several useful features. For example, fitting 204 can comprise an angle between 0 and 90 degrees, and preferably between 30 and 60 degrees, and even more preferable an angel of about 45 degrees. The rigidness of fitting 204 combined with the angle of the fitting prevents tubing 202 from pulling cannula 100 down, making it easier to achieve a more effective coupling between cannula 100 and the patient's nose.

As can also be seen, fitting 204 comprises a tab 205a that mates with, or protrudes through, window 108 to achieve a tight coupling between tube 202 and cannula 100. Fitting 204 also comprise a second tab 206a that can be used to push fitting 204 into end portion 104a. The other end of fitting 204 can be configured such that it can push fit into tube 202.

A cannula plug 207 can be configured to interface with the opposite end of cannula 100. As can be seen, plug 207 can also comprise a tab 205b and a tab 206b, which operate similarly to tabs 205a and 206b. As mentioned previously, the sides of the cannula 100 are interchangeable, so that the tube fitting 204 may be connected to either end 104a/104b of the cannula, and the cannula plug 207 would be connected to the opposite side.

Conventional cannulas comprise wing tabs at either end of the cannula where end portions 104a and 104b are on the cannula 100 of the current disclosure. The wing tabs allow, e.g., the tubing to be affixed to the cannula by looping a corresponding strap on the tube through holes or slits on the wing tab. But such conventional methods of affixing the tub to the cannula do not create the tight coupling possible via cannula 100.

A head strap 208 can be configured to attach to fitting 204 and plug 207, such that the cannula can be strapped to the patient's head. For example, the ends of strap 208, fitting 204, and plug 207 can be configured such that the ends of strap 208 can be lopped through and secured to the ends of fitting 204 and plug 207. The radius of curvature of cannula 100 is such that when the strap 208 is tightened, there is minimum deformation of cannula 100.

The other end of tube 202 from the interface with cannula 100 can, e.g., be configured to push fit with a conical snap fitting 210, which can also, e.g., push fit with a conical fitting 212. Chain link 214 and collar clip 216 can be configured to allow tubing 202 to be affixed to the patient's collar.

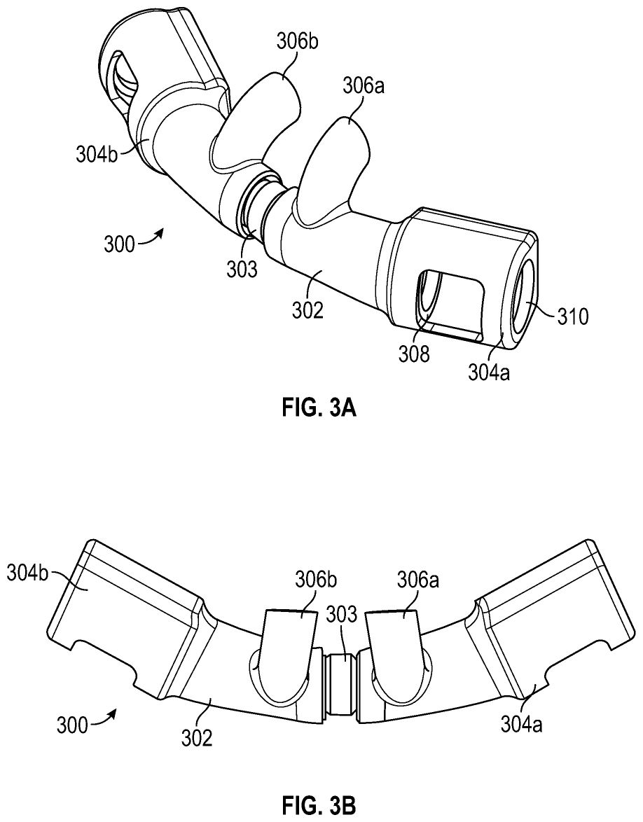

FIG. 3 illustrates a cannula 300 in accordance with another example embodiment. As can be seen cannula 300 comprises similar features as those of cannula 100, which are denoted with similar reference numbers. A difference between cannula 300 and cannula 100 is the collapsible portion 303 of body 302, which can also be seen in the top view of FIG. 3B. Collapsible portion 303 can be collapsed and expanded through a plurality of positions by pushing on the ends of cannula 300. The collapsible portion 303 allows for additional flex for a more aggressive angular contouring without buckling or kinking.

The collapsible portion 303 is shown in FIG. 3A as being in the middle of the cannula 300 (between the prongs 306a and 306b), however, the collapsible portion 303 may be located anywhere along the cannula (for example, to one side of both prongs). Furthermore, multiple collapsible portions may be included in the cannula 300.

FIG. 3C illustrates an embodiment of the cannula 320 that is similar to the cannula 300 of FIGS. 3A and 3B, except that cannula 320 includes three collapsible portions 303a/303b/303c. In other embodiments, the cannula of the present disclosure can include any one or two of the collapsible portions 303a/303b/303c shown in FIG. 3C.

FIG. 3D illustrates an embodiment of the cannula 340 that is similar to the cannula 300 of FIGS. 3A and 3B except that the prongs 306a and 306b are collapsible for greater adjustability. As shown in FIG. 3D, the prongs 306a and 306b include collapsible portions 307a and 307b. In various embodiments, the collapsible prongs of FIG. 3D may be combined with any of the features described in other cannula embodiments of the present disclosure. For example, the cannula may include multiple collapsible portions on its body and the collapsible prongs.

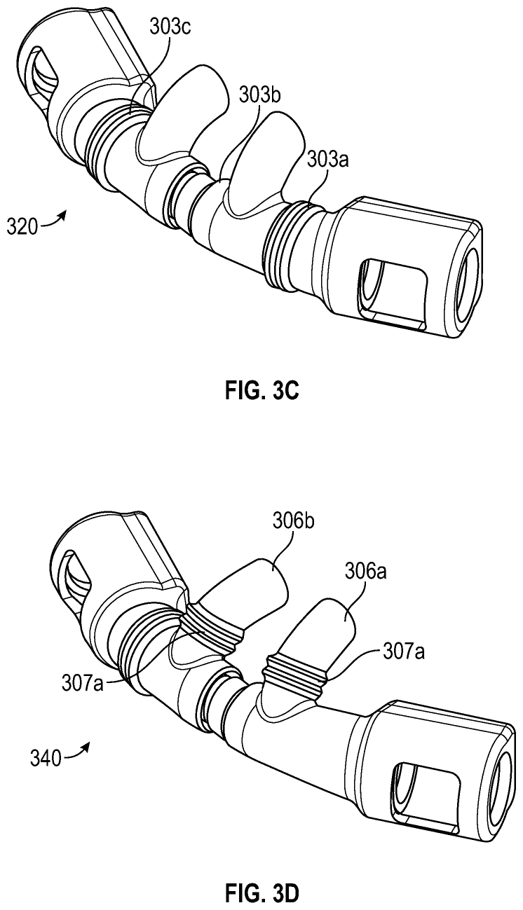

FIG. 4 illustrates a tubing assembly 400 that is similar to tubing assembly 200, but including cannula 300. As can be seen in FIG. 4, cannula 300 can also be configured to be used with an adjustable frame 414 to help hold the position of the cannula 300. The frame 414 provides additional structural integrity for cannula 300. The ends 416a and 416b of frame 414 can be configured to interface with the rest of assembly 400, e.g., via an interface or coupling with the tabs (not shown) of fitting 404 and plug 407 that interface with windows 308a and 308b of cannula 300. The coupling mechanism can be a zip tie, hook and loop, quick disconnect, adjustable strap, plastic clip, etc.

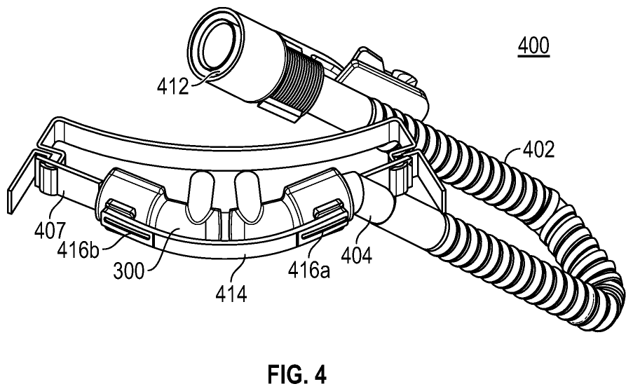

FIG. 5 illustrates a cannula 500 in accordance with another example embodiment. As can be seen cannula 500 comprises similar features as those of cannulas 100, 300, 320, and 340, which are denoted with similar reference numbers. A difference between cannula 500 and cannula 100 is the bellows portion 507 of body 502. Bellows portion 507 can be collapsed and expanded as need to fit the patient as the strap 608 is tightened. The bellows portion 507 allows for additional flex for a more aggressive angular contouring without buckling or kinking. The bellows portion 507 may be located in the middle of the body 502 (as shown in FIG. 5) or anywhere throughout the cannula. In addition, multiple bellows portions 507 may be included along the cannula. In some embodiments, the cannula prongs 506a/506b may include bellows for adjustability. Embodiments of the cannula 500 having multiple bellows 507, prong bellows portions, and/or non-centrally located bellows 507 may be similar to embodiments of the cannula shown in FIGS. 3C and 3D (with the bellows portions and prong bellows portions replacing the collapsible portions and collapsible prongs portions, respectively).

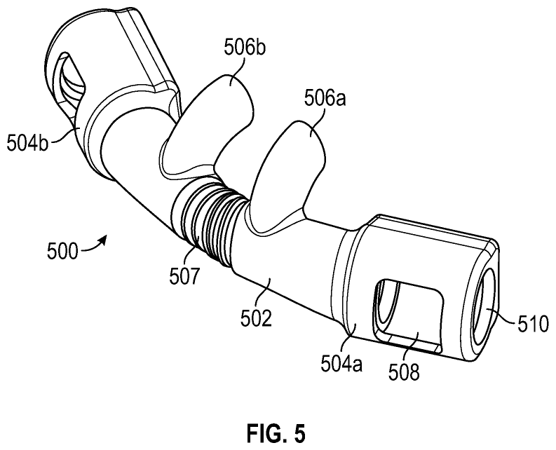

FIG. 6 illustrates a tubing assembly 600 that is similar to tubing assembly 200 and 400, but including cannula 500. As can be seen in FIG. 6, cannula 500 can also be configured to be used with an adjustable frame 614 to help hold the position of the cannula 500. The frame 614 provides additional structural integrity for cannula 500. The ends 616a and 616b of frame 614 can be configured to interface with the rest of assembly 600, e.g., via an interface or coupling with the tabs (not shown) of fitting 604 and plug 607 that interface with windows 508a and b of cannula 500. The coupling mechanism can be a zip tie, hook and loop, quick disconnect, adjustable strap, plastic clip, etc.

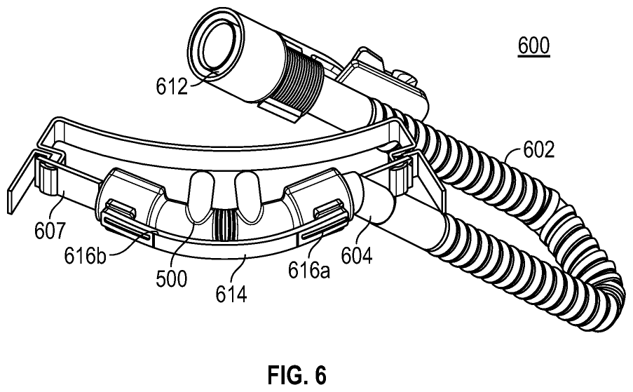

FIG. 7A illustrates a cannula 700 in accordance with another example embodiment. As can be seen, cannula 700 comprises similar features as those of cannulas 100, 300 (320, 340), and 500, which are denoted with similar reference numbers. A difference between cannula 700 the other cannulas is that a frame 714 (e.g., a flexible metal frame) can be insertable, snap-fit, pressed-fit, directly molded, etc., with body 702, which can also be seen in the top view of FIG. 7B. A mid-portion 709 can comprise a collapsible portion 303 or a bellows portion 507, which allows for additional flex for a more aggressive angular contouring without buckling or kinking. Frame 714 can then be pinch, formed and reformed to various shapes. FIGS. 7A and 7B show the frame 714 fitted to the front of the cannula, however, the frame may be fitted/molded to other parts of the cannula (e.g., front, top, bottom sides).

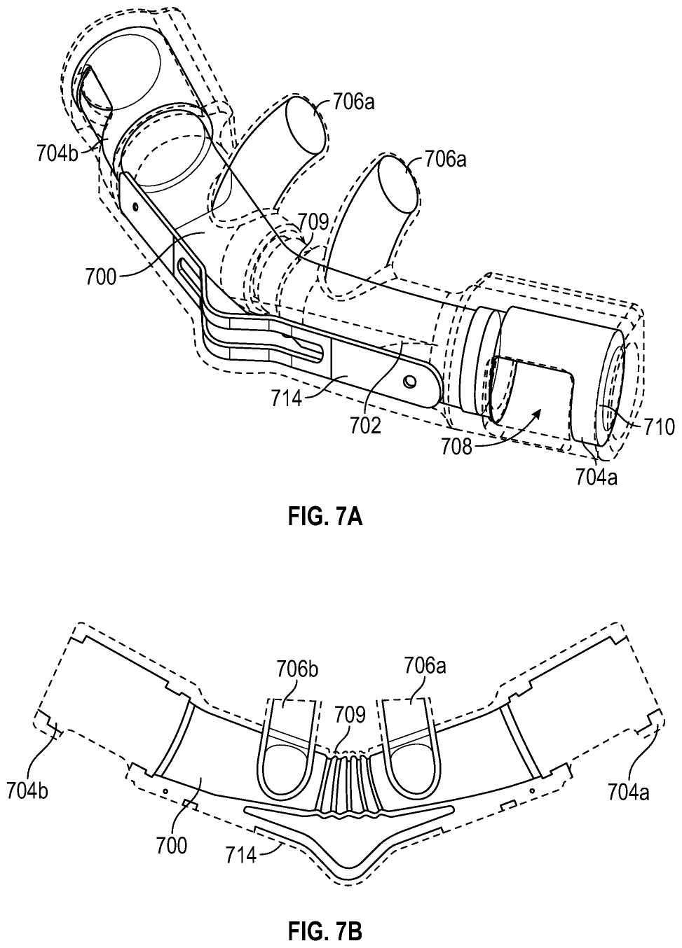

FIG. 8 shows an example of a cannula 800, which may include features of any of the cannulas 100, 300, 320, 340, 500 described above, except that a frame 814 (e.g., a flexible metal frame) is fitted to the back of the cannula. Similar to frame 714, the frame 814 can be insertable, snap-fit, pressed-fit, directly molded, etc., with body of the cannula 800. The frame 814 can be pinch, formed, and reformed to various shapes. FIG. 8 shows the frame 814 fitted to the back of the cannula, however, the frame may be fitted/molded to other parts of the cannula (e.g., front, top, bottom sides).

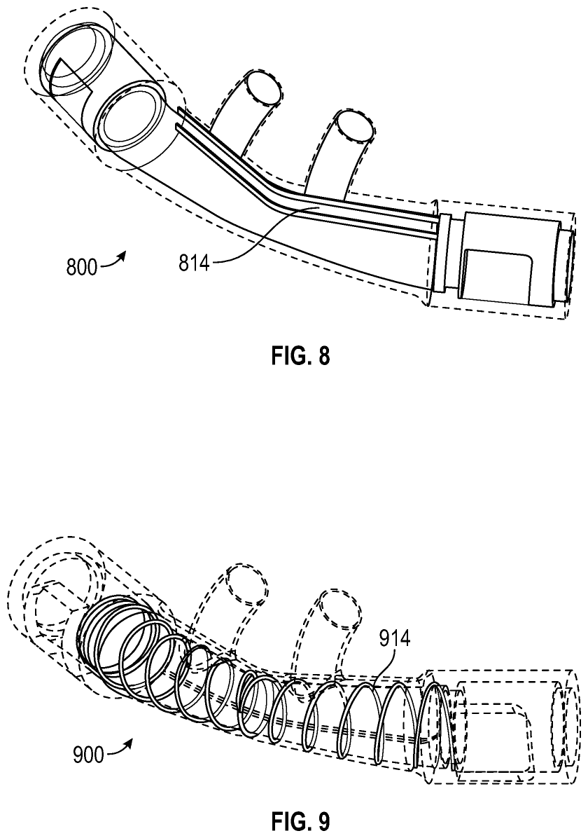

FIG. 9 shows an example of a cannula 900, which may include features of any of the cannulas 100, 300, 320, 340, 500 described above, except that a spring or coil frame 914 is fitted around the body of the cannula 900. The coil frame 914 may be, for example, a flexible metal coil with coils sized to fit snugly to the shape of the cannula (as shown in FIG. 9). The coil frame 914 may cover the entire length of the body of the cannula (as shown in FIG. 9) or, alternatively, cover a part of the length (e.g., a middle portion).

It will be understood that the benefits and advantages described above may relate to one embodiment or may relate to several embodiments. Aspects described in connection with one embodiment are intended to be able to be used with the other embodiments. Any explanation in connection with one embodiment applies to similar features of the other embodiments, and elements of multiple embodiments can be combined to form other embodiments. The embodiments are not limited to those that solve any or all of the stated problems or those that have any or all of the stated benefits and advantages.

The preceding detailed description is merely exemplary in nature and is not intended to limit the possible embodiments or the application and uses thereof. The described embodiments are not limited to usage in conjunction with a particular type of flow generation system. Hence, although the present embodiments are, for convenience of explanation, depicted and described as being implemented with ventilators, it will be appreciated that it can be implemented in various other types of air flow systems and machines with similar types of cannulas, and in various other systems and environments. Furthermore, there is no intention to be bound by any theory presented in any preceding section. It is also understood that the illustrations may include exaggerated dimensions and graphical representation to better illustrate the referenced items shown, and are not consider limiting unless expressly stated as such.

What is claimed is:

1. An air flow cannula, comprising:
a body having a radius of curvature less than about 180 degrees, the body including two solid portions and an adjustable portion between the two solid portions,
two nasal posts, and
two end portions.

2. The air flow cannula of claim 1, wherein the radius of curvature is greater than about 90 degrees.

3. The air flow cannula of claim 1, wherein each of the two end portions comprises a hole for receiving a cannula tube fitting.

4. The air flow cannula of claim 3, wherein each of the two end portions further comprises a window that can accommodate a tab on the cannula tube fitting for effective coupling of the cannula tube fitting with the respective end portion.

5. The air flow cannula of claim 1, wherein the two end portions are configured to facilitate attaching tubing at either end of the air flow cannula.

6. The air flow cannula of claim 1, further comprising: an adjustable frame attached to the body and configured to provide additional structural integrity to the air flow cannula.

7. The air flow cannula of claim 6, wherein the adjustable frame is inserted into the body, snap-fit to the body, pressed-fit to the body, or directly molded in the body.

8. The air flow cannula of claim 1, wherein the adjustable portion is configured to collapse to a plurality of positions.

9. The air flow cannula of claim 1, wherein the adjustable portion is configured to collapse and expand as needed.

10. The air flow cannula of claim 1, wherein the adjustable portion is a bellows portion.

11. A tube system comprising:
tubing;
a head strap;
an air flow system cannula, comprising:
a body having a radius of curvature less than about 180 degrees, the body comprising two solid portions and an adjustable portion between the two solid portions,
two nasal posts, each of the two nasal posts protruding from a respective one of the two solid portions of the body, and
two end portions, each of the two end portions comprising a hole and a window;
a cannula plug comprising a first cannula plug tab configured to engage the window in one of the two end portions, a second cannula plug tab configured to enable the cannula plug to be pushed into the window of the same end portion, and a coupling mechanism for coupling the cannula plug with the head strap; and
a cannula tube fitting comprising a first cannula tube fitting tab at a first end of the cannula tube fitting configured to engage the window in the other of the two end portions, a second cannula tube fitting tab configured to enable the cannula tube fitting to be pushed into the window of the same end portion, a coupling mechanism for coupling the cannula plug with the head strap, and a second end of the cannula tube fitting configured to couple with the tubing.

12. The system of claim 11, wherein the radius of curvature is greater than about 90 degrees.

13. The system of claim 11, wherein the adjustable portion is configured to collapse to a plurality of positions for additional flex and angular contouring.

14. The system of claim 13, wherein each of the plurality of positions is a discrete position.

15. The system of claim 13, wherein the adjustable portion is a bellows portion.

16. The system of claim 11, further comprising an adjustable frame attached to the body and configured to provide additional structural integrity to the body.

17. The system of claim 16, wherein the adjustable frame is attached to the two solid portions of the body.

18. The system of claim 16, wherein the adjustable frame is configured to be inserted into the body, snap-fit to the body, pressed-fit to the body, or directly molded in the body.

19. The system of claim 16, further comprising a coupling mechanism for coupling the adjustable frame to the body, wherein the comprising mechanism is one of a zip tie, hook and loop, quick disconnect, adjustable strap, or plastic clip.

20. The system of claim 19, wherein the coupling mechanism is configured to engage with the first cannula plug tab or the first cannula tube fitting tab.
